(19) United States
(12) Patent Application Publication
(10) Pub. No.: US 2021/0010022 A1
(43) Pub. Date: Jan. 14, 2021

(54) NOVEL NUCLEIC ACID CONSTRUCT

(71) Applicants: CAMBRIDGE ENTERPRISE LIMITED, Cambridge, Cambridgeshire (GB); GENOME RESEARCH LIMITED, Cambridge, Cambridgeshire (GB)

(72) Inventors: William SKARNES, Cambridge (GB); Bonkyoung KOO, Cambridge (GB); Juergen FINK, Cambridge (GB); Jihoon KIM, Cambridge (GB); Alessandra MERENDA, Cambridge (GB); Camelia Roxana MICSIK, Cambridge (GB); Amanda Maria Hei-Ran ANDERSSON-ROLF, Cambridgeshire (GB)

(73) Assignees: Cambridge Enterprise Limited, Cambridge, Cambridgeshire (GB); Genome Research Limited, Cambridge, Cambridgeshire (GB)

(21) Appl. No.: 16/304,859

(22) PCT Filed: May 26, 2017
(86) PCT No.: PCT/GB2017/051500

§ 371 Date: Nov. 27, 2018

(30) Foreign Application Priority Data
May 27, 2016 (GB) 1609439.3
Aug. 12, 2016 (GB) 1613839.8

(51) Int. Cl. C12N 15/85; C12N 9/22; A01K 67/027; C12N 15/90; C12N 5/074

(52) U.S. Cl. CPC C12N 15/8509; C12N 9/22; A01K 67/0276; C12N 15/907; C12N 2800/30; C12N 2310/20; A01K 2217/075; A01K 2227/105; C12N 5/0696

(57) ABSTRACT

The invention relates to a nucleic acid construct for bi-allelic conditional modification of a target gene and methods of use thereof.

### FIELD OF THE INVENTION

The invention relates to a nucleic acid construct for bi-allelic conditional modification of a target gene and methods of use thereof.

### BACKGROUND OF THE INVENTION

Analysing gene function is a crucial step in our understanding of normal physiology and disease pathogenesis. In cell models, loss-of-function studies require inactivation of both copies of the gene. Prior to the development of site-specific nucleases, gene knockouts in cell lines were achieved by loss-of-heterozygosity (Yusa, K. et al. (2004) Nature 429, 896-9) or serial gene targeting approaches (Niwa, H. et al. (2000) Nat. Genet. 24, 372-6). The development of site-specific nucleases, such as zinc finger nucleases, has greatly facilitated functional studies in cells due to the fact that both copies of a gene can be efficiently inactivated in a single step (Bibikova, M. et al. (2001) Mol. Cell. Biol. 21, 289-97; Bibikova, M. et al. (2002) Genetics 161, 1169-75; Zou, J. et al. (2009) Cell Stem Cell 5, 97-110; Kim, H. J. et al. (2009) Genome Res. 19, 1279-88; Perez, E. E. et al. (2008) Nat. Biotechnol. 26, 808-16). Recently, CRISPR-Cas9 gene editing technology (Cho, S. W. et al. (2013) Nat. Biotechnol. 31, 230-2; Cong, L. et al. (2013) Science 339, 819-23; Jinek, M. et al. (2013) Elife 2, e00471; Mali, P. et al. (2013) Science 339, 823-826) has become the tool of choice for gene knockout studies due to its simplicity and robustness. Cas9 nuclease is an RNA-guided nuclease that is highly efficient in inducing a double-stranded break (DSB) at a genomic site of interest, often observed on both chromosomes. These DSBs can be repaired by the error-prone non-homologous end joining (NHEJ) pathway to generate gene-inactivating mutations or, in the presence of a donor template, the DSBs can be repaired by homology-directed repair (HDR) to generate more precise and more complex alleles (Cho, S. W. et al. (2013) supra; Cong, L. et al. (2013) supra; Jinek, M. et al. (2013) supra; Mali, P. et al. (2013) supra).

While simple constitutive knockouts are useful and informative, in many cases it is desirable to engineer conditional loss-of-function models, particularly for essential genes required for cell viability or embryonic development. Conditional strategies are well-established in mouse models to study the function of genes at a specific developmental stage or in a tissue-specific manner. Conditional alleles are designed to eliminate gene function through the action of site-specific recombinases, such as Cre recombinase. In general, this involves the introduction of two recombinase recognition sites (e.g. loxP) flanking a critical exon(s) of the target gene (Testa, G. et al. (2004) Genesis 38, 151-8; Skarnes, W. C. et al. (2011) Nature 474, 337-42) and the inclusion of a drug selection marker for homologous recombination in mouse embryonic stem cells. An alternative method, COIN (conditional made by inversion) (Economides, A. N. et al. (2013) Proc. Natl. Acad. Sci. U.S.A. 110, E3179-88) has been developed that involves the use of a ‘flippable’ reporter gene and a drug selection marker inserted into an intron or an exon of a gene by homologous recombination. Conditional gene inactivation is achieved by first removing the drug selection cassette to ensure proper expression of the target gene and then reversing the orientation of the reporter cassette with Cre recombinase to block transcription of the target gene and activate reporter gene expression. With either strategy, extensive breeding of animals is required first to remove the selection cassette and to generate animals homozygous for the conditional (floxed) allele that also carry a Cre transgene.

More recently, CRISPR-Cas9 has simplified the engineering of conditional alleles in animal models by enabling the generation of floxed alleles directly in zygotes (Wang, H. et al. (2013) Cell 153, 910-918; Yang, H. et al. (2013) Cell 154, 1370-9). In addition, strategies based on inducible expression of Cas9 have been developed for conditional mutagenesis of genes in cells and in animal models (Gonzalez, F. et al. (2014) Cell Stem Cell 15, 215-26; Dow, L. E. et al. (2015) Nat. Biotechnol. 33, 390-4; Zetsche, B., et al. (2015) Nat. Biotechnol. 33, 139-142). This method, however, depends on bi-allelic mutation of the target gene by error-prone NHEJ and therefore lacks precision. Bi-allelic modification of cells by

NHEJ will produce mixtures of cells with undefined genotypes (frameshift and in-frame indels), complicating the phenotyping of mutant cells.

There is therefore a need to provide bi-allelic conditional gene modifications that is able to overcome the problems associated with currently available methods.

### SUMMARY OF THE INVENTION

According to a first aspect of the invention, there is provided the use of a nucleic acid construct for bi-allelic conditional modification of a target gene, wherein said construct is an artificial intron comprising:
(a) an expression cassette in antisense orientation relative to the target gene;
(b) one or more pairs of recombinase sites, wherein at least one pair flanks the expression cassette; and
(c) one or more components that inactivate the target gene, such that following exposure to a recombinase the expression cassette inverts and the target gene is inactivated.

According to a second aspect of the invention, there is provided a nucleic acid construct which is an artificial intron comprising a splice donor at one end, a first branch point and a first splice acceptor at the other end, which additionally comprises:
(a) an expression cassette positioned between the splice donor and first branch point, said expression cassette comprising a promoter, an open reading frame and a 3′ untranslated region, each of which is in antisense orientation relative to the first splice donor, branch point and splice acceptor;
(b) a first pair of recombinase sites, the first of which is positioned between the splice donor and the 3′ untranslated region of the expression cassette and the second of which is positioned between the first splice acceptor and the first branch point;
(c) a second branch point and second splice acceptor, each of which is positioned between the promoter and open reading frame of the expression cassette and is in antisense orientation relative to the first splice donor, branch point and splice acceptor; and
(d) a second pair of recombinase sites which flank the open reading frame, 3′ untranslated region, second splice acceptor and second branch point, such that following exposure to a recombinase, the orientation of said first pair of recombinase sites causes inversion of said expression cassette and results in one recombinase site from the first pair of recombinase sites and one recombinase site from the second pair of recombinase sites being orientated to cause excision of the promoter and first branch point.

According to a further aspect of the invention, there is provided the use of the nucleic acid construct as defined herein for conditional gene modification.

According to a further aspect of the invention, there is provided a method of conditional gene modification, comprising:
(a) co-transfection of a double-strand break-inducing agent, a gene targeting agent and the nucleic acid construct as defined herein into a cell;
(b) selection of a cell wherein at least one allele comprises the nucleic acid construct;

and
(c) exposing the cell as defined in step (b) to a recombinase.

### BRIEF DESCRIPTION OF THE FIGURES

FIG. 1: FLIP cassette strategy for bi-allelic conditional gene modification. a. Schematic drawing of the FLIP cassette strategy for bi-allelic conditional gene modification. The Cas9 nuclease is directed to the genomic site of interest by the gRNA where it generates a double stranded break (DSB, left panel). This DSB can be repaired by non-homologous end joining (NHEJ) generating insertions/deletions (indels) or homology directed repair (HDR, right panel). In the latter, the donor plasmid is used as a template for precise correction of the DSB and thus facilitating insertion of the FLIP cassette in the genome. Bi-allelic conditional gene modification is achieved when one allele is repaired via NHEJ generating a frame shift mutation and the other through HDR, resulting in FLIP cassette insertion.
b. The design of the FLIP cassette. The FLIP cassette contains several elements: i) a reporter or resistance gene which expression is controlled by a promoter and polyadenylation signal (initially in the antisense direction) ii) two pairs of loxP sites for Cre mediated recombination iii) splicing donor and acceptor sites (including two branching points) for spliceosome recognition and intron excision. The FLIP cassette is flanked with left and right homologous arms, each arm less than 1 kb, generating a targeting vector. Following Cre recombination the cassette is inverted (flipped) and a new splicing configuration is activated. This results in inactivation of the gene via three rearrangements: the old splice site is disrupted (BP1 removed), the inversion of the pA and new splice (BP2) signal into the sense direction leads to termination of transcription. SD—splice donor, SA1, SA2—splice acceptor, loxP sites—grey and dark grey triangles, BP1, BP2 (circles)—branching point, pA—polyadenylation signal.
c. The FLIP cassette containing a DsRed reporter gene was inserted into the cDNA of eGFP as an artificial intron and transfected in HEK 293T cells.
d. Following insertion, the cassette functions as an intron and does not disrupt the expression of the eGFP cDNA. Hence, both eGFP and DsRed proteins are expressed (top row). After Cre treatment the eGFP expression is disrupted, and only DsRed expression is maintained (bottom row). Scale bar 400 μm.

FIG. 2: Insertion of the FLIP cassette in the endogenous Ctnnbl gene of mouse embryonic stem cells.
a. The FLIP cassette containing a resistance gene was inserted into the 5th exon of Ctnnbl. SD—splice donor, SA—splice acceptor, grey and dark grey triangles—loxP site, BP—branching point.
b. PCR detection of FLIP cassette insertion in the Ctnnbl locus. Correctly targeted clones E2, B1, and G12 are positive for 5′ and 3′arm genotyping PCR reactions (for genotyping strategy see FIG. 4b). Exon 5 PCR detects the remaining allele. The clones (E2, B1 and G12) are correctly targeted.
c. Insertions/deletions in the non-targeted allele were identified by Sanger sequencing. Clone B1 has a 1 base pair (bp) insertion and clone G12 has a 164 bp deletion. gRNA and PAM recognition sequences are represented in light and dark grey respectively.

The predicted wild type (VVT) sequence is shown in a grey box and the actual sequence of the second allele (not having a FLIP cassette inserted) is aligned underneath.

d and e. Detection of β-catenin protein by immunofluorescence (d) and western blotting (e) before and after Cre transfection. This confirmed the loss of β-catenin at protein level for the FLIP/−clones, B1 and G12 following Cre treatment.

f. Representative bright field images of the ESC clones before (top) and after (bottom) Cre transfection. B1 and G12 clones displayed altered phenotype due to Ctnnb1 gene inactivation.

FIG. 3: Summary of targeting using CRISPR-FLIP strategy.

FIG. 4: Step-wise Cre recombination and inversion of the FLIP cassette.
a. Inversion (flipping) of the FLIP cassette. Schematic showing the step wise recombination of loxP sites following Cre treatment. Following the first recombination the loxP sites represented by grey triangles (left) or the loxP sites represented by dark grey triangles (right) will be recombined. As the loxP sites are facing each other the result is an inversion. During the second recombination the loxP sites, now aligned in the same direction recombine. The result is deletion of the PGK promoter and branch point 1 (BP1). SD—splice donor, SA—splice acceptor, grey and dark grey triangles—loxP site, BP—branching point.
b. Genotyping strategy used to confirm clones targeted with the FLIP cassette. The arrows represent primers, and the primer pairs are colour coded. The drawing shows the position of the primers in the genome and in the FLIP cassette. The dark grey (5′) and grey (3′) primers were used to confirm correct integration of the FLIP cassette. The allele not having integrated a FLIP cassette but potentially sustained indels due to NHEJ is genotyped and sequenced with the primer pair represented by the arrows.

FIG. 5: Workflow

Representative image of the workflow including time estimate for generating bi-allelic conditional KOs using the CRISPR-FLIP technology.

FIG. 6: Validation of the FLIP cassette by insertion in the endogenous Esrrb and Sox2 genes.

Detection of Correctly Targeted Esrrb Clones
a. The FLIP cassette containing a resistance gene was inserted into the 2nd exon of Esrrb.
b. Detection of correctly targeted Esrrb clones. Detection of correctly integrated 5′arm and 3′arms by PCR in ESC clones targeted with the FLIP cassette.
c. The clones G11, B3 and H5 are correctly targeted. Sequencing results of the second allele of the Esrrb gene allow identification of insertions/deletions.
d. Clone B3 has a 5 base pair (bp) deletion and clone H5 has a 34 bp deletion. Loss of protein expression following Cre treatment was confirmed by Western blot. Detection of correctly targeted Sox2 clones
e. The FLIP cassette containing a resistance gene was inserted into the exon of Sox2.
f. Detection of correctly integrated 5′arm and 3′arms by PCR in ESC clones targeted with the FLIP cassette. The clones A2, HOM are correctly targeted. Sequencing results of the second allele of the Sox2 gene allow identification of insertions/deletions, this was used to confirm the FLIP/+genotype of clone A2. The lack of a wt band confirms the genotype of the HOM FLIP/FLIP clone.
g and h. Loss of protein following Cre treatment (gene inactivation) was confirmed by immunofluorescence (g) and Western blot (h). Please note that in this case a homozygous FLIP/FLIP clone was used to show the loss of protein expression and functionality of the FLIP cassette.

FIG. 7: Validation of the FLIP cassette by insertion in the endogenous Apc, Tcf712 and Trim37 genes.

Detection of Correctly Targeted Apc Clones
a. The FLIP cassette containing a resistance gene was inserted into the 16 th exon of Apc.
b. Detection of correctly integrated 5′arm and 3′arms by PCR in ESC clones targeted with the FLIP cassette. The clones A3, D5 are correctly targeted.
c. Sequencing results of the second allele of the Apc gene allow identification of insertions/deletions. Clone D5 has a 10 bp deletion.

Detection of Correctly Targeted Nfx1 Clones
d. The FLIP cassette containing a resistance gene was inserted into the 2 nd exon of Nfx .
e. Detection of correctly integrated 5′arm and 3′arms by PCR in ESC clones targeted with the FLIP cassette.
f. The clones E1, F6 are correctly targeted. Sequencing results of the second allele of the MO gene allow identification of insertions/deletions (f). Clone F6 has a 22 bp deletion.

Detection of Correctly Targeted Tcf7/2 Clones
g. The FLIP cassette containing a resistance gene was inserted into the 5th exon of Tcf7/2.
h. Detection of correctly targeted Tcf7/2 clones. Detection of correctly integrated 5′arm and 3′arms by PCR in ESC clones targeted with the FLIP cassette.
i. The clones C3, A6, A11 are correctly targeted. Sequencing results of the second allele of the Tcf7/2 gene allow identification of insertions/deletions. Clone A6 has a 10 bp deletion and A11 has a 1 bp deletion.

Detection of Correctly Targeted Trim13 Clones
j. The FLIP cassette containing a resistance gene was inserted into the 3 rd exon of Trim13.
k. Detection of correctly integrated 5′arm and 3′arms by PCR in ESC clones targeted with the FLIP cassette. The clones H3, H4, G10 are correctly targeted.
l. Sequencing results of the second allele of the Trim gene allow identification of insertions/deletions (bottom right and left). Clone H3 has a 2 bp insertion and G10 has a 1 bp deletion.

Detection of Correctly Targeted Trim37 Clones
m. The FLIP cassette containing a resistance gene was inserted into the 6 th exon of Trim37.
n. Detection of correctly integrated 5′arm and 3′arms by PCR in ESC clones targeted with the FLIP cassette.
o. The clones E3, H5, F11 are correctly targeted. Sequencing results of the second allele of the Trim37 gene allow identification of insertions/deletions. Clone H5 has a 13 bp deletion and Fll has a 4 bp deletion.

SD—splice donor, SA—splice acceptor, grey and dark grey triangles—loxP site, BP—branching point, pA—polyadenylation signal, gRNA and PAM recognition sequences are represented in grey and dark grey respectively.

FIG. 8: Detection of correctly targeted human ARID1A (hARID1A) in human embryonic kidney cells 293 (HEK293) clones.
a. The FLIP cassette containing a resistance gene was inserted into the 3rd exon of hARID1a.
b. Detection of correctly integrated 5′arm and 3′arms by PCR in HEK293 clones targeted with the FLIP cassette.
c. The clones Fl, F8, B8 are correctly targeted. Sequencing results of the second allele of the hARID1A gene allow identification of insertions/deletions. Clone F8 has a 5 bp deletion and clone B8 has a 47 bp deletion.

Detection of Correctly Targeted Human TP53 (hTP53) in Human Embryonic Kidney Cells 293 (HEK293) Clones
d. The FLIP cassette containing a resistance gene was inserted into the 4 th exon of hTP53.
e. Detection of correctly integrated 5′arm and 3′arms by PCR in HEK293 clones targeted with the FLIP cassette.
f. The clones D1, E2, D6 are correctly targeted. Sequencing results of the second allele of the hTP53 gene allow identification of insertions/deletions. Clone E2 has a 19 bp deletion and clone D6 is homozygous for the FLIP cassette

FIG. 9: Detection of correctly targeted human TP53 (hTP53) in human induced pluripotent stem cell (hiPSC) clones.
a. The FLIP cassette containing a resistance gene was inserted into the 4th exon of hTP53.
b. Detection of correctly integrated 5′arm and 3′arms by PCR in hiPSC clones targeted with the FLIP cassette.
c. The clones H4, C4, F4 are correctly targeted. Sequencing results of the second allele of the hTP53 gene allow identification of insertions/deletions. Clone C4 has an 11 bp deletion and clone F4 has a 13 bp insertion.

FIG. 10: Reversible conditional gene inactivation with FLIP-FIpE (FLIP-FIlp Excision) intronic cassette.
a. The FLIP-FIpE cassette containing a DsRed reporter gene was inserted into the cDNA of eGFP as an artificial intron and transfected in HEK 293T cells. The FLIP-FIpE cassette contains the same elements as the FLIP cassette except the addition of two FRT sites flanking the region containing the cryptic splice acceptor and pA. SD—splice donor, SA1, SA2—splice acceptor, grey and dark grey triangles—loxP sites, ovals—FRT sites, BP1, BP2 (circles)—branching point, pA—polyadenylation signal.
b. Following insertion, the cassette functions as an intron and does not disrupt the expression of the eGFP cDNA. Hence, both eGFP and DsRed proteins are expressed (top row). After Cre recombination the eGFP expression is disrupted, and only DsRed expression is maintained (bottom row). Following FIp recombination, the mutagenic cassette is excised and the eGFP expression is restored.
c. The FLIP-FIpE cassette containing a resistance gene was inserted into the 5th exon of Ctnnbl. SD—splice donor, SA—splice acceptor, grey and dark grey triangles—loxP site, ovals—FRT sites, BP1, BP2 (circles)—branching point.
d. PCR detection of FLIP-FIpE cassette insertion in the Ctnnbl locus. The correctly targeted clone (A8) is positive for 5′ and 3′arm genotyping PCR reactions. Exon 5 PCR detects the remaining allele.
e. Insertions/deletions in the non-targeted allele were identified by Sanger sequencing. Clone A8 has a 1 base pair (bp) insertion. gRNA and PAM recognition sequences are represented in blue and purple respectively. The predicted wild type (VVT) sequence is shown in the grey box and the actual sequence of the second allele (not having a FLIP cassette inserted) is aligned underneath. Clone A8 is correctly targeted with FLIP-FIpE in one allele and a+1 frameshift mutation in the other allele.
f and g. Detection of β-catenin protein by immunofluorescence (f) and western blotting (g) in control, Cre induction and Cre and FIp dual induction. The loss of β-catenin on protein level is evident after Cre recombination and it can be restored by FIp recombination.
h. Representative bright field images of the A8 clone in control, Cre induction and Cre and FIp dual induction. Cre-mediated gene inactivation results in altered phenotype due to the loss of Ctnnb1 gene. FIp recombination restores the gene expression and cells regain their original dome-shaped morphology.

### DETAILED DESCRIPTION OF THE INVENTION

According to a first aspect of the invention, there is provided the use of a nucleic acid construct for bi-allelic conditional modification of a target gene, wherein said construct is an artificial intron comprising:
(a) an expression cassette in antisense orientation relative to the target gene;
(b) one or more pairs of recombinase sites, wherein at least one pair flanks the expression cassette; and
(c) one or more components that inactivate the target gene,

such that following exposure to a recombinase the expression cassette inverts and the target gene is inactivated.

The use as described herein is a simplified, one-step method for engineering conditional loss-of-function mutations in diploid cells. The inventors have developed a novel invertible drug selection cassette, FLIP, for high-efficiency nuclease-assisted targeting in cells, such that they are able to recover bi-allelic events with a single round of gene targeting and screening. As proof-of-principle, the inventors conditionally inactivate genes in mouse and human embryonic stem cells including essential genes for their self-renewal.

In one embodiment, the bi-allelic conditional modification of a target gene is reversible.

Nucleic Acid Construct

According to a second aspect of the invention, there is provided a nucleic acid construct which is an artificial intron comprising a splice donor at one end, a first branch point and a first splice acceptor at the other end, which additionally comprises:
(a) an expression cassette positioned between the splice donor and first branch point, said expression cassette comprising a promoter, an open reading frame and a 3′ untranslated region, each of which is in antisense orientation relative to the first splice donor, branch point and splice acceptor;
(b) a first pair of recombinase sites, the first of which is positioned between the splice donor and the 3′ untranslated region of the expression cassette and the second of which is positioned between the first splice acceptor and the first branch point;
(c) a second branch point and second splice acceptor, each of which is positioned between the promoter and open reading frame of the expression cassette and is in antisense orientation relative to the first splice donor, branch point and splice acceptor; and
(d) a second pair of recombinase sites which flank the open reading frame, 3′ untranslated region, second splice acceptor and second branch point, such that following exposure to a recombinase, the orientation of said first pair of recombinase sites causes inversion of said expression cassette and results in one recombinase site from the first pair of recombinase sites and one recombinase site from the second pair of recombinase sites being orientated to cause excision of the promoter and first branch point.

Described herein is an invertible drug selection cassette for high-efficiency nuclease-assisted targeting in cells, able to recover bi-allelic events with a single round of gene targeting and screening.

The inventors further modified the cassette to generate a reversible conditional allele, the benefits of which include the application of ‘switchable’ gene expression. Therefore, in one embodiment, the nucleic acid construct additionally comprises:
(e) a third pair of recombinase sites, wherein said third pair of recombinase sites are distinct from the first and second pair of recombinase sites and flank the open reading frame and 3′ untranslated region of the expression cassette, second splice acceptor and second branch point,

such that following exposure to a recombinase, the orientation of said third pair of recombinase sites causes excision of the open reading frame and 3′ untranslated region of the expression cassette, second splice acceptor and second branch point.

Reference to the term “nucleic acid construct” as used herein, refers to an artificially synthesised nucleic acid sequence comprising non-specific and specific sequences of nucleic acids. A nucleic acid construct may also be known as an insert or a cassette.

Nucleic acid sequences provided by this invention can be assembled from cDNA fragments and short oligonucleotide linkers, or from a series of oligonucleotides, to provide a synthetic construct which is capable of being inserted in a recombinant expression vector and expressed in a recombinant transcriptional unit. Alternatively, they may be synthesized partially or in their entirety.

Reference to the term “artificial intron” as used herein, refers to a nucleic acid sequence comprising the features of an intron. Such features include, but are not limited to, a splice donor, a branch point and a splice acceptor. In one embodiment, the branch point is upstream of the splice acceptor, such as more than 10 bp upstream of the splice acceptor, in particular between 30 and 70 bp upstream of the splice acceptor. In a further embodiment, the branch point is 46 bp upstream of the splice acceptor. In an alternative embodiment, the branch point is 56 bp upstream of the splice acceptor. In a further embodiment, the splice donor is upstream of the branch point and splice acceptor. Therefore, reference to the term “intronic cassette” as used herein refers to a nucleic acid construct comprising an artificial intron.

In one embodiment, the splice donor of the nucleic acid construct described herein comprises the following sequence GTAAG.

In one embodiment, the splice acceptor of the nucleic acid described herein comprises the following sequence TTTCCCTCCCTTAG (SEQ ID NO. 1).

In one embodiment, the branch point of the nucleic acid construct described herein comprises the following sequence CTGAT or CTGAC.

Reference to the term “expression cassette” as used herein, refers to a nucleic acid construct comprising a gene that is operably linked to suitable transcriptional regulatory elements. Such regulatory elements include, but are not limited to, a transcriptional promoter, an optional operator sequence to control transcription, an open reading frame, sequences which control the termination of transcription and a 3′ untranslated region.

Promoters include, but are not limited to, constitutively active promoters (SV40, CAGG, UBC, EF1a, CMV, PGK), tissue-specific or development-stage-specific promoters, inducible promoters (chemically or physically regulated promoters) and synthetic promoters. In one embodiment, the promoter is a constitutively active promoter. In a further embodiment, the promoter is the mouse phosphoglycerate kinase 1 (PGK) promoter. In still a further embodiment, the promoter comprises the following sequence SEQ ID NO. 2.

Reference to the term “open reading frame” as used herein, refers to the part of a nucleic acid sequence that has the potential to code for a protein or peptide. In one embodiment, the open reading frame encodes one or more selectable markers. In a further embodiment, the open reading frame encodes one or more negative and/or positive selectable markers. In a further embodiment, the open reading frame encodes one or more negative selectable marker. In an alternative embodiment, the open reading frame encodes one or more positive selectable marker. In still a further embodiment, the one or more selectable markers encode a fusion protein.

In a further embodiment, the open reading frame comprises a reporter gene and/or a drug resistance gene. In still a further embodiment, the open reading frame comprises a reporter gene. Examples of reporter genes include, but are not limited to, lacZ (encoding β-galactosidase), luc (encoding luciferase), gfp (encoding green fluorescent protein) and associated alternatives, uidA (encoding β-glucuronidase) and alkaline phosphatase associated reporters. In a further embodiment, the open reading frame comprises dsRed2 reporter gene. In still a further embodiment, the open reading frame comprises the following sequence SEQ ID NO. 3.

In an alternative embodiment, the open reading frame comprises a relevant drug resistance gene. In a further embodiment, the open reading frame comprises an antibiotic resistance gene. Examples of drug resistance genes include, but are not limited to, genes encoding resistance to kanamycin, spectinomycin, streptomycin, ampicillin, carbenicillin, bleomycin, erythromycin, polymyxin B, tetracycline, chloramphenicol, blasticidin, G418/geneticin, hydromycin B, zeom and puromycin. It will be appreciated that one skilled in the art will be adept at selecting an appropriate drug resistance gene taking into account the target cell. Therefore, in a further embodiment, the open reading frame comprises a mammalian specific gene, such as a gene encoding resistance to blasticidin, G418/geneticin, hygromycin B, zeocin or puromycin. In a further embodiment, the open reading frame comprises a puromycin resistance gene (puroR). In still a further embodiment, the open reading frame comprises the following sequence SEQ ID NO. 4.

The nucleic acid construct may contain an expression cassette composed of a promoter driving an antibiotic resistance gene thus enriching for cells that undergo homologous recombination of one allele and NHEJ damage on the second allele, following exposure to a double-strand break-inducing agent. Upon exposure to a recombinase the nucleic acid construct is inverted into a mutagenic configuration leading to a complete loss of gene function in the cell. As a consequence of the inversion, a cryptic splicing signal is activated for the target gene inactivation and is further ensured by a termination signal and the disruption of the first splice acceptor.

Reference to the term “3′ untranslated region” as used herein, refers to a nucleic acid sequence which is transcribed but not translated, wherein said nucleic acid sequences comprise regulatory regions that post-transcriptionally influence gene expression. Regulatory regions within the 3′ untranslated region can influence polyadenylation, translation efficiency, localization, and stability of the transcript. The 3′ untranslated region may comprise both binding sites for regulatory proteins as well as microRNAs (miRNAs). The 3′ untranslated region may also comprise silencer regions which bind to repressor proteins and inhibit the expression of the mRNA. 3′ untranslated regions may also comprise AU-rich elements (AREs). Proteins bind AREs to affect the stability or decay rate of transcripts in a localized manner or affect translation initiation. Furthermore, the 3′ untranslated region may comprise nucleic acid sequences that direct the addition of adenine residues to the end of the mRNA transcript, often called the poly(A) tail. Poly(A) binding protein (PABP) binds to this tail and contributes to regulation of mRNA translation, stability, and export. The 3′ untranslated region may also comprise sequences that attract proteins to associate the transcript with the cytoskeleton, transport it to or from the cell nucleus, or perform other types of localization. In addition to sequences within the 3′-UTR, the physical characteristics of the region, including its length and secondary structure, may contribute to translation regulation. Therefore, in one embodiment, the 3′ untranslated region comprises a transcriptional termination signal, such as microRNA response elements, ARE rich elements and/or a polyadenylation signal, such as a polyadenylation tail and/or alternative polyadenylation. In a further embodiment, the transcriptional termination signal comprises a polyadenylation signal. In still a further embodiment, the transcriptional termination signal comprises a polyadenylation tail. In still a further embodiment, the transcriptional termination signal comprises the following sequence SEQ ID NO. 5.

In one embodiment, the nucleic acid construct as described herein comprises one homologous arm. In an alternative embodiment, the nucleic acid construct as described herein comprises two homologous arms. In one embodiment, the homologous arm or arms are individually more than 20 bp, such as between 50 and 100 bp. In an alternative embodiment, the homologous arm or arms are individually more than 100 bp, such as between 200 and 1000 bp.

The position of the homologous arm relative to the double-strand break and any additional requirements of a functional homologous arm will be known to one skilled in the art. For example, for optimal splicing insertion points that match the consensus sequence for mammalian splice junctions (minimally MAGR (^{A}/_{C}AG/Pu) and at least AGR (AG/Pu)) may be selected, such as between AG and Pu. In one embodiment, the homologous arm is designed so that the insertion sites of the modification are less than 500 bp away from the double-strand break, such as less than 100 bp away, in particular less than 15 bp away.

In one embodiment, the nucleic acid construct described herein comprises the following sequence SEQ ID NO 6. In an alternative embodiment, the nucleic acid constrict described herein comprises the following sequence SEQ ID NO. 7.

In one embodiment, the nucleic acid as described herein is incorporated within a vector. Examples of suitable vectors include, but are not limited to, plasmids, bacteriophages, viruses and artificial chromosomes. In a further embodiment, the nucleic acid as described herein is incorporated within a mammalian expression vector, such as pCDNA4TO vector. In an alternative embodiment, the nucleic acid as described herein is incorporated within a pUC118 vector.

In one embodiment, the nucleic acid construct described herein is incorporated within a vector and comprises the following sequence SEQ ID NO. 8.

In an alternative embodiment, the nucleic acid construct described herein is incorporated within a vector and comprises the following sequence SEQ ID NO. 9.

In one embodiment, the nucleic acid construct is downstream of a promoter. In a further embodiment the nucleic acid construct is downstream of a CMV promoter.

In one embodiment the nucleic acid construct is within a reporter gene. In a further embodiment the nucleic acid construct is within an eGFP gene.

Recombination

Site-specific recombination, also known as conservative site-specific recombination, is a type of genetic recombination in which DNA strand exchange takes place between segments possessing at least a certain degree of sequence homology. Site-specific recombinases (SSRs) perform rearrangements of DNA segments by recognizing and binding to recombinase sites, at which they cleave the nucleic acid backbone, exchange the two nucleic acid segments involved and rejoin the strands. While in some site-specific recombination systems just a recombinase enzyme and the recombination sites are enough to perform all these reactions, in other systems a number of accessory proteins and/or accessory sites are also needed.

Examples of recombinases include, but are not limited to, lambda-integrase, φC31-integrase, Cre-recombinase, FLP-recombinase, gamma-delta-resolvase, Tn3-resolvase, Dre recombinase, VCre recombinase, and SCre recombinase. These enzymes mediate DNA rearrangements including integration, excision/resolution and inversion along different reaction routes based on their origin and architecture. Therefore, in one embodiment, the recombinase is able to integrate/excise and/or invert a DNA sequence. Based on amino acid sequence homology and mechanism of action most site-specific recombinases are grouped into one of two families, the tyrosine recombinase family or the serine recombinase family. Therefore, in a further embodiment, the recombinase is a serine recombinase. In an alternative embodiment, the recombinase is a tyrosine recombinase.

Cre (“causes recombination”) is able to recombine specific sequences of DNA without the need for cofactors. The enzyme recognizes loxP (“locus of crossover in phage P1”) sites, and depending on the orientation of these recombinase sites with respect to one another, Cre will integrate/excise or invert DNA sequences. Upon the excision (called “resolution” in case of a circular substrate) of a particular DNA region, normal gene expression is considerably compromised or terminated. Therefore, in a further embodiment, the recombinase is Cre-recombinase.

Due to the pronounced resolution activity of Cre, one of its initial applications was the excision of loxP-flanked (“floxed”) genes leading to cell-specific gene knockout of such a floxed gene after Cre becomes expressed in the tissue of interest. Current technologies allow for both the spatial and temporal control of Cre activity. Such methods facilitating the spatial control of genetic alteration often involve the selection of a tissue-specific promoter to drive Cre expression and allows for the localized expression of Cre in certain tissues. In order to control temporal activity of the excision reaction, forms of Cre which take advantage of various ligand binding domains have also been developed. One successful strategy for inducing specific temporal Cre activity involves fusing the enzyme with a mutated ligand-binding domain for the human estrogen receptor (ERt). Upon the introduction of tamoxifen (an estrogen receptor antagonist), the Cre-ERt construct is able to penetrate the nucleus and induce Cre-mediated recombination. ERt binds tamoxifen with greater affinity than endogenous estrogens, which allows Cre-ERt to remain cytoplasmic in animals untreated with tamoxifen.

Flippase, or FLP-recombinase, is a tyrosine family site-specific recombinase which recognizes FRT sites.

It will be understood that the term “recombinase site” as used herein, refers to a sequence of one or more nucleic acids which interact with a recombinase. Examples of recombinase sites include, but are not limited to, Rox, VloxP, SloxP, attP, attB, loxP and FRT sites. In one embodiment, the first and/or second recombinase sites comprise loxP sites. In a further embodiment, the third pair of recombinase sites comprise FRT sites. It would be understood by one skilled in the art that different combinations of recombinases and associated recombinase sites are within the scope of the invention described herein and may be suitable for different methodologies.

LoxP (locus of X(cross)-over in P1) sites comprise 34-base-pair long sequences consisting of two 13-bp long palindromic repeats separated by an 8-bp long asymmetric core spacer sequence. The asymmetry in the core sequence gives the loxP site directionality, and the canonical loxP sequences are known in the art. The loxP sequence does not occur naturally in any known genome other than P1 phage, and is long enough that there is virtually no chance of it occurring randomly, Therefore, inserting loxP sites at deliberate locations in a DNA sequence allows for very specific manipulations. Therefore, in a further embodiment, the first pair of recombinase sites and/or second pair of recombinase sites each comprise the following sequence SEC) ID NO. 10 and/or SEQ ID NO. 11.

FRT (flippase recognition target) sites comprise a 34-base-pair long sequence consisting of two 13-bp arms flanking a 8-bp long asymmetric core spacer. Several variant FRT sites exist, but recombination can usually occur only between two identical FRTs. Examples of FRT sites will be known to one skilled in the art.

As described herein, a consequence of the inversion is the activation of a cryptic splicing signal for inactivation of the target gene, which is further ensured by a transcription termination signal and the disruption of the first splice acceptor. Therefore, in one embodiment, following exposure to a recombinase said second branch point and second splicing acceptor are orientated to cause productive splicing with the first splice donor.

Conditional Gene Modification

Existing methods for engineering conditional mutations in cultured cells rely on the inclusion of a drug selection cassette that must be removed in a second step to ensure proper expression of targeted conditional alleles. These methods were not designed for the generation of conditional loss-of-function models in a single step, particularly where the target gene is essential for cell growth or viability.

To overcome these limitations, the strategy presented herein combines an invertible intronic cassette (FLIP), similar to COIN, with high efficiency Cas9-assisted gene editing. The non-mutagenic orientation of the FLIP cassette expresses the puromycin resistance gene (puroR) to select for correct nuclease-assisted targeting into the exon of one allele and simultaneous enrichment of cells that inactivate the second allele by nuclease-mediated NHEJ (FIG. 1a). Upon exposure to Cre recombinase the FLIP cassette is inverted to a mutagenic configuration that activates a cryptic splice acceptor and polyadenylation signal (pA) and disrupting the initial splicing acceptor resulting in the complete loss of gene function (FIG. 1b and FIG. 4). In contrast to COIN, which requires the removal of the drug selection cassette, our FLIP cassette permits the generation of conditional mutant cells in one step.

According to a further aspect of the invention, there is provided the use of the nucleic acid construct as defined herein for conditional gene modification.

Reference to the term “conditional modification” as used herein, refers to a modification that has no functional effect under certain (permissive) environmental conditions and a functional effect under other (restrictive) conditions. For example, a conditional mutation refers to a mutation that presents a wild type phenotype under permissive environmental conditions and a mutant phenotype under restrictive conditions. The FLIP intronic cassette when targeted into an exon is ignored by the splicing machinery and preserves normal expression of the target gene (FIG. 1a).

In one embodiment, the conditional gene modification is reversible.

Reference to the term “wild type” as used herein, refers to proteins, peptides, amino acid and nucleotide sequences which are present in nature.

Reference to the term “allele” as used herein, refers to a variant form of a gene. Some genes have a variety of different forms, which are located at the same position, or genetic locus, on a chromosome. Humans are called diploid organisms because they have two alleles at each genetic locus, with one allele inherited from each parent. Each pair of alleles represents the genotype of a specific gene. Genotypes are described as homozygous if there are two identical alleles at a particular locus and as heterozygous if the two alleles differ. Alleles contribute to the organism's phenotype, which is the outward appearance of the organism.

In one embodiment, said conditional gene modification is bi-allelic. In a further embodiment, the conditional gene modification is bi-allelic and reversible.

It would be understood that the gene modification as presented herein is directly applicable to a variety of diploid organisms and may also be adapted to organisms of different ploidy, in particular multiploid or aneuploid cell lines and tetraploid organisms.

According to a further aspect of the invention, there is provided a method of conditional gene modification, comprising:
(a) co-transfection of a double-strand break-inducing agent, a gene targeting agent and the nucleic acid construct as defined herein into a cell;
(b) selection of a cell wherein at least one allele comprises the nucleic acid construct; and
(c) exposing the cell as defined in step (b) to a recombinase specific for the first and/or second pair of recombinase sites.

In one embodiment, the method additionally comprises:
(d) exposing the cell to a further recombinase specific for the third pair of recombinase sites.

In one embodiment, the cell as defined in step (a) is a diploid cell, such as a mammalian cell, in particular a human or mouse derived cell. In an alternative embodiment, the cell as defined in step (a) is an aneuploid cell, in particular a stably immortalised human cell line, such as HEK293.

In one embodiment, the selection as defined in step (b) is of a cell wherein the first allele comprises the nucleic acid construct and the second allele comprises a gene-inactivating mutation and/or the nucleic acid construct.

In one embodiment, the selection in step (b) comprises confirmation of correct integration of the nucleic acid construct as described herein and/or confirmation of non-homologous end joining events in the second allele.

In a further embodiment, the selection in step (b) comprises use of the expression cassette and/or polymerase chain reaction and/or sequencing. In still a further embodiment, the selection in step (b) comprises first selection of drug resistant and/or fluorescent colonies.

Reference to the term “gene targeting agent” as used herein, refers to an agent that defines the genomic target to be modified. Gene targeting agents include, but are not limited to, gRNA. In one embodiment, said gene targeting agent is gRNA.

Reference to the term “gRNA” as used herein, may refer to short synthetic RNA composed of a scaffold sequence necessary for Cas9-binding and a user-defined nucleotide spacer or targeting sequence. In one embodiment, the gRNA binds to or close to the putative intron insertion site. It will be known that the consensus sequence for mammalian splice junctions is minimally MAGR (^{A}/_{C}AG/Pu) and at least AGR (AG/Pu).

It would be understood that methods of transfection include, but are not limited to, chemical-based methods (cyclodextrin, polymers, liposomes, nanoparticles), non-chemical methods (electroporation, cell squeezing, sonoporation, optical transfection, impalefection, hydrodynamic, heat shock), particle-based methods (magnetofection, particle bombardment) and viral methods. In one embodiment, exposing the cell as defined in step (b) to a recombinase, comprises transfection. In a further embodiment, exposing the cell as defined in step (b) to a recombinase, comprises chemical-based transfection, in particular comprising liposomes.

Reference to the term “co-transfection” as used herein, refers to the simultaneous transfection with two or more separate nucleic add molecules. In one embodiment, co-transfection of a double-strand break-inducing agent, a gene targeting agent and the nucleic acid construct as defined herein into a cell is via chemical-based and/or non-chemical based transfection. In a further embodiment, co-transfection of a double-strand break-inducing agent, a gene targeting agent and the nucleic acid construct as defined herein into a cell is via chemical-based transfection. In an alternative embodiment, co-transfection of a double-strand break-inducing agent, a gene targeting agent and the nucleic acid construct as defined herein into a cell is via non-chemical based transfection, such as electroporation.

Reference to the term “double-strand break-inducing agent” as used herein, refers to an agent that breaks both DNA strands. Example include, but are not limited to, exogenous agents (radiation, chemical agents) and endogenous agents (reactive oxygen species).

DNA double strand breaks are made when two complementary stands of the DNA double helix are broken simultaneously at sites that are sufficiently close to one another that base-pairing and chromatin structure are insufficient to keep the two DNA ends juxtaposed. As a consequence, the two DNA ends generated by a double strand break are liable to become physically dissociated from one another, making ensuing repair difficult to perform and providing the opportunity for inappropriate recombination with other sites in the genome.

In one embodiment, said double-strand break inducing agent is selected from TALENs, zinc finger nucleases and Cas9. Using CRISPR/Cas9 technology, the FLIP cassette is introduced into an exon and contains splicing signals that allow the targeted gene to be functionally transcribed. Therefore, in a further embodiment, said double-strand break inducing agent is Cas9.

The strategy adopted by the inventors for the generation of conditional loss-of-function cell models combines a drug-selectable invertible cassette (FLIP), similar to COIN (Economides, A. N. et al. (2013) supra), with high efficiency Cas9-assisted gene editing by homology directed repair.

DNA double strand breaks in mammalian cells are primarily repaired by homologous recombination (HR) and non-homologous end joining (NHEJ). NHEJ is referred to as “non-homologous end joining” because the break ends are directly ligated without the need for a homologous template, in contrast to homology directed repair such as HR, which requires a homologous sequence to guide repair. Inappropriate NHEJ may lead to indels (insertion or deletion of bases in the DNA) that can generate frameshift mutations.

In one embodiment, the gene inactivating mutation is an indel-mediated frameshift or truncation mutation. In a further embodiment, the indel is a product of non-homologous end joining.

The project leading to this application has received funding from the European Research Council (ERC) under the European Union's Horizon 2020 research and innovation programme (Grant Agreement No. 639050). The following studies and protocols illustrate embodiments of the methods described herein:

Materials and Methods

dsRed FLIP Cassette Inserted in the eGFP cDNA

The FLIP cassette inserted in the middle of eGFP and containing a dsRed2 reporter gene was synthesized and ordered from GenScript. The split eGFP cDNA and the FLIP cassette were cloned into the mammalian expression vector pCDNA4TO (Invitrogen) using BamHl (R0 136S, NEB) and Xhol (R0 146S, NEB) (for pre-recombined form). The vector was subsequently transformed into Cre expressing bacteria (A111, Gene bridges) to generate the Cre-recombined form. Correct clones were confirmed with restriction digest BamHl (R0 136S, NEB) and Xhol (R0 146S, NEB) and Sanger sequencing. The FLIP-FIpE cassette was also synthesized and inserted into the same site of the eGFP expression vector.

FLIP Cassette Containing Selection Marker Genes

The FLIP cassette was PCR amplified using primers Flip_UniL (SEQ ID NO. 12) and Flip_UniR (SEQ ID NO. 13) and cloned into Pjet1.2 vector (ThermoFisher Scientific, K131). Replacement of dsRed was done through restriction digest excision using EcoRl (R3101S, NEB) and Acc65I (R0599S, NEB) followed by insertion of PCR amplified selection marker genes, amplified from plasmids using primers Puro-L-Acc651 (SEQ ID NO.14) and Puro-R-EcoRl (SEQ ID NO. 15), Blast-L-Acc651 (SEQ ID NO. 16) and Blast-R-EcoRl (SEQ ID NO. 17), using Infusion cloning (638909, Clontech). The FLIP cassette including selection marker gene was then amplified and prepared before being transferred to the vector pUC118 (3318, Clontech) using the restriction enzymes Sacl (R0 156S, NEB) and Pstl (R0 140S, NEB) and Mighty cloning (6027, Takara).

Addition of Homologous Arms to the FLIP Cassette—FLIP Targeting Vector Generation

Homologous arms around an intron insertion site were amplified by high fidelity Phusion DNA polymerase (M0 530S, NEB). After PCR product purification, both homologous arms and FLIP cassette-containing vector were mixed with a type II restriction enzyme and T4 DNA ligase (M0 202T, NEB). After 25 cycles of 37° C. and 16° C., the reaction mixture was directly used for E. coli transformation. DNA was extracted (27106, Qiagen) and analysed with restriction digest to identify correctly assembled FLIP donor vectors.

Cas9 and gRNA Plasmids

Human codon optimized Cas9 (41815, Addgene) and empty gRNA vector (41824, Addgene) were obtained from Addgene.

Cell Culture Conditions

HEK293 cells

Human embryonic kidney 293 cells were cultured in media consisting of DMEM, high glucose (11965092, Thermofisher Scientific) supplemented with 10% foetal bovine serum (Thermofisher Scientific), lx penicillin-streptomycin according to the manufacturer's recommendation (P0781, Sigma). The cells were tested negative for mycoplasma.

Embryonic Stem Cells (ESCs)

Murine E14 Tg2a embryonic stem (mES) cells were cultured feeder-free on 0.1% gelatin-coated dishes in serum+LIF+2i (Chiron and PD03) composed of GM EM (G5154, Sigma), 10% foetal bovine serum (Gibco), lx non-essential amino acids according to the manufacturer's recommendation (11140, Thermofisher Scientific), 1mM sodium pyruvate (113-24-6, Sigma), 2 mM L-glutamine (25030081, Thermofisher Scientific), lx penicillin-streptomycin according to the manufacturer's recommendation (P0781, Sigma) and 0.1 mM 2-mercaptoethanol (M7522, Sigma), 20 ng/ml murine LIF (Hyvonen lab, Cambridge), 3 μM CHIR99021 and 1 pM PD0325901 (Stewart lab, Dresden). mES cells were kept in a tissue culture incubator at 37° C. and 5% CO₂. Cells were split in a 1:10-1:15 ratio every 3-4 days depending on confluence. All cells were tested negative for mycoplasma.

Cell Transfections

For targeting of ESCs 1×10⁶ cells were collected and resuspended in magnesium and calcium free phosphate buffered saline (D8537, Sigma). A total of 50μg of DNA consisting of the targeting vector, Cas9 and gRNA in a 1:1:1 ratio were added to the cells and then transferred to a 4mm electroporation cuvette (Biorad). Electroporation was performed using the Biorad Gene Pulser XCell's (165-2660, Biorad) exponential program and the following settings: 240V, 500uF, unlimited resistance. For targeting of human iPS cells, 2×10⁶ cells were dissociated with Accutase (SCR005, Millipore) and resuspended in nucleofection buffer (Solution 2, LONZA). A total of 12 μg of DNA consisting of 4 μg Cas9 plasmid, 4 μg of each gRNA plasmid and 4 μg of targeting vector was added to the cells and transferred to a 100 μl nucleofection cuvette (LONZA). Nucleofection was performed with the AMAXA Human Nucleofector Kit 2 (LONZA Cat #VPH-5022) using the B-016 program. The cells were plated and cultured for 1 day in TeSR-E8 media containing ROCK inhibitor (Y-27632, Stem Cell Technologies) to promote survival of transfected cells.

For targeting of HEK293 cells, the cells were cultured until they reached 50-60% confluence. A total of 8 μg of DNA consisting of targeting vector, Cas9 and gRNA in a 1:1:1 ratio was transfected using Lipofectamine 2000 (11668019, Invitrogen) according to the manufacturer's instructions.

Cre and FIp Transfection

1 μg of pCAGGS-Cre-IRES-Puro and/or pCAGGS-FIp-IRES-Puro plasmid vector and 3 μl of Lipofectamine2000 (Invitrogen) were mixed according to the manufacturer's protocol, applied to 200.000 cells/6-well and incubated overnight. Media was refreshed the following morning.

Western Blot

Following transfection ESCs were cultured for 2-5 days and then lysed in buffer containing complete protease-inhibitor cocktail tablets (11697498001, Roche) and centrifuged at 13000 rpm for 15min at 4° C. Protein concentration was measured with Bradford assay (5000204, Biorad) and equal amounts were loaded on a 10% acrylamide gel and run at 120V for 1.5-2 hrs. The proteins were subsequently transferred to an lmmobilon-FL PVDF 0.45 μm membrane (IPFL00010, Millipore) at 90V for 1hr 15min. The following primary antibodies and dilutions were used to detect the indicated proteins: Rabbit monoclonal antibody against β-Catenin (1:1000, 8480S, Cell Signaling), mouse monoclonal against alpha Tubulin antibody (1:5000, ab7291, Abcam), mouse monoclonal antibody against Esrrb, (1:1000, PP-H6705-00, Bio-Techne) rat monoclonal antibody against Sox2, (1:500, 14-9811-80, eBioscience), and rabbit monoclonal against vinculin (1:3000, ab19002, Abcam). The membrane was washed and the indicated horseradish-peroxidase conjugated secondary antibodies were applied: horse anti-mouse IgG (1:5000, Cell Signaling) and goat anti-rabbit (1:5000, Cell Signaling) and goat anti-rat HRP conjugated (1:5000, SC2032, Santa Cruz). Detection was achieved using ECL prime Western blotting Detection system (RPN2133, GE Healthcare).

lmmunofluorescence

Cells were cultured in Ibid tissue culture dishes (IB-81156, Ibid) coated with 0.1% gelatin, washed twice with calcium and magnesium free PBS and fixed in 4% PFA for 20min at RT. The cells were permeabilised in 0.5% Triton X-100 (T8787, Sigma) in PBS for 15min at RT. Subsequently, blocking was performed in 5% donkey serum (D9663, Sigma) and 0.1% Triton X-100 for 1 hr at RT. The following primary antibodies in blocking buffer were applied for the indicated protein: Sox2, (1:500, 14-9811-80, eBioscience) and β-Catenin (1:1000, 4627, Cell Signaling). Primary antibodies were incubated overnight at 4° C. Subsequently excess primary antibody was washed away and anti-rat Alexa Flour 594® conjugated antibody (1:1000, A21209, Abcam) was added for Sox2, and incubated for 1h at RT. Excess secondary antibody was washed away and DAPI (1:1000, D9542, Sigma) was added and incubated for 10 min at RT. Cells were washed and mounted in RapiClear (RCCS002, Sunjin lab).

Acknowledgements

pCAGGS-Cre-IRES-Puro and pCAGGS-FIp-IRES-Puro plasmid vectors were kindly provided by B. Hendrich (WT-MRC Cambridge Stem Cell Institute, UCAM).

### EXAMPLE 1

### FLIP dsRed2 Cassette

Using CRISPR/Cas9 technology, the FLIP cassette is introduced into an exon and contains splicing signals that allow the targeted gene to be functionally transcribed. Critically, the FLIP cassette contains a selectable marker composed of the PGK promoter driving the puromycin resistance (puroR) gene thus enriching for cells that undergo Cas9-mediated homologous recombination of one allele and NHEJ damage on the second allele. Upon exposure to Cre recombinase the FLIP cassette is inverted into a mutagenic configuration leading to a complete loss of gene function in the cell. As a consequence of the inversion, a cryptic splicing signal is activated for the target gene inactivation and is further ensured by a polyadenylation (pA) signal and the disruption of original splice acceptor (FIG. 1b).

Initially, to test the functionality of our intronic FLIP cassette, the inventors constructed a FLIP cassette variant containing a dsRed2 reporter in place of puroR into a CMV-eGFP (enhanced green fluorescent protein) expression plasmid (FIG. 1c). Following transient transfection of HEK293 cells, both green and red fluorescence was observed, demonstrating that insertion of the FLIP cassette in the non-mutagenic orientation is inert (FIG. 1d). Cre recombinase mediated FLIP cassette inversion resulted in loss of eGFP expression, showing conditional inactivation of eGFP expression in the inverted, mutagenic orientation (FIG. 1c,d).

### EXAMPLE 2

### Bi-Allelic Conditional Modification

The inventors then employed CRISPR/Cas9 endonuclease in mouse embryonic stem cells (mESCs) to introduce the puroR FLIP cassette into one allele of β-catenin (Ctnnb1) via HDR and to simultaneously induce a frameshift mutation by NHEJ in the second β-catenin allele (FIG. 1a, 2a). β-catenin is an important gene for the morphology and efficient self-renewal of mESCs (Anton, R. et al. (2007) FEBS Lett. 581, 5247-5254; Lyashenko, N. et al. (2011) Nat. Cell Biol. 13, 753-61). A donor vector containing the puroR FLIP cassette inserted in exon 5 of β-catenin and flanked by ˜1 kb homology arms was transfected into mESCs with Cas9 and gRNA expression plasmids. Following selection in puromycin, drug-resistant colonies were genotyped by PCR to confirm correct integration of the FLIP cassette and then assayed for NHEJ events in the second allele by Sanger sequencing (FIG. 2b,c, FIG. 4b).

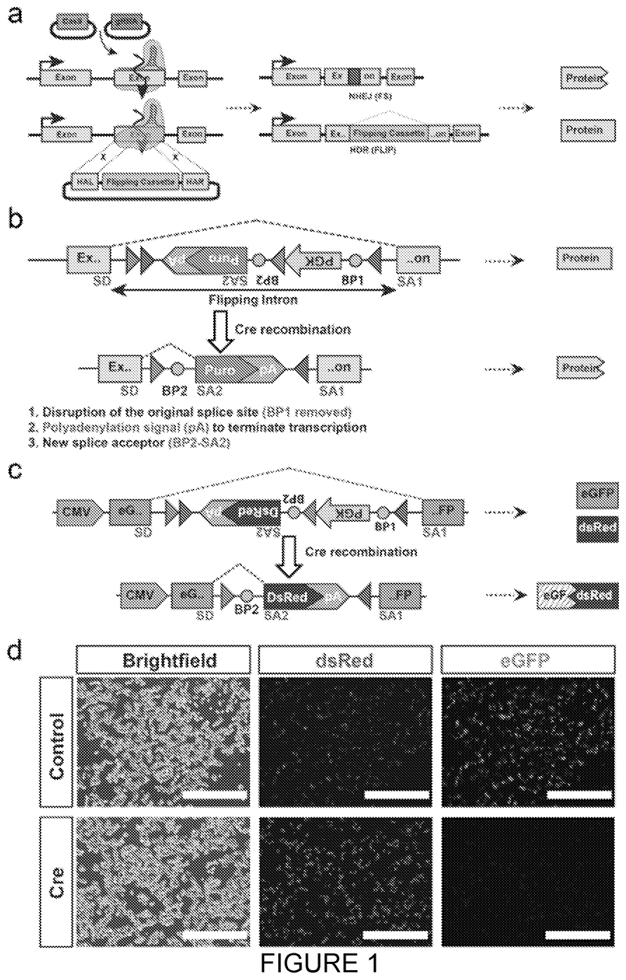

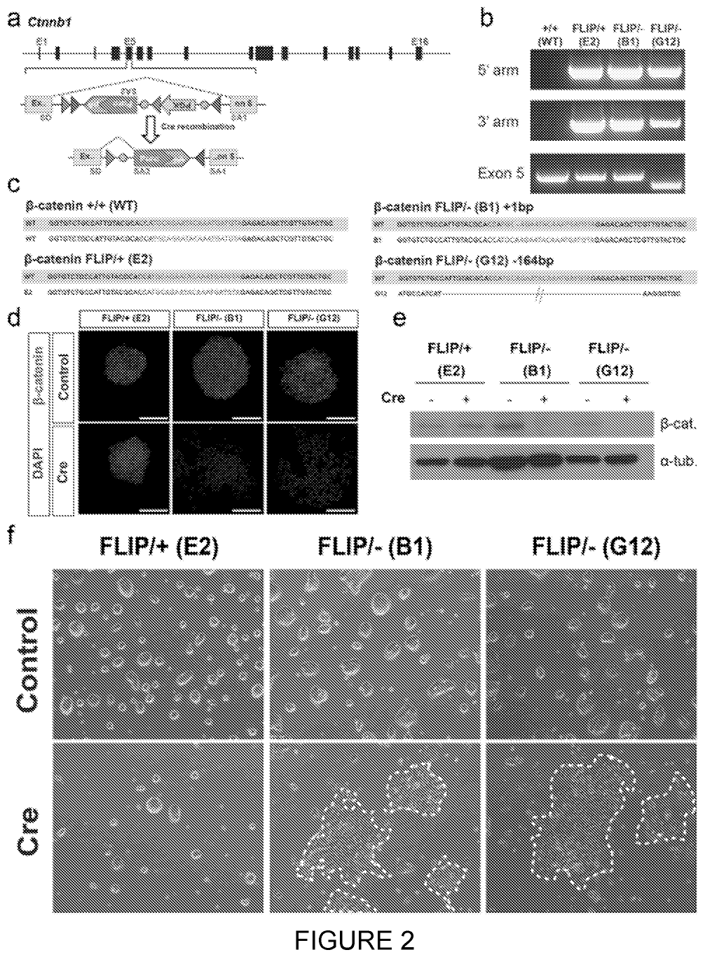

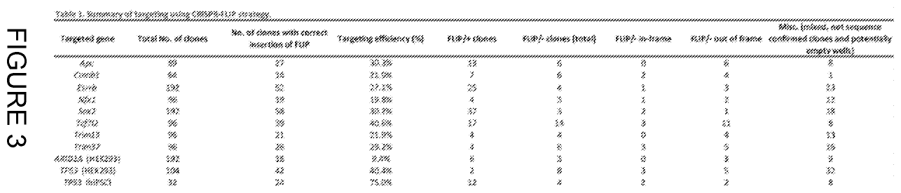

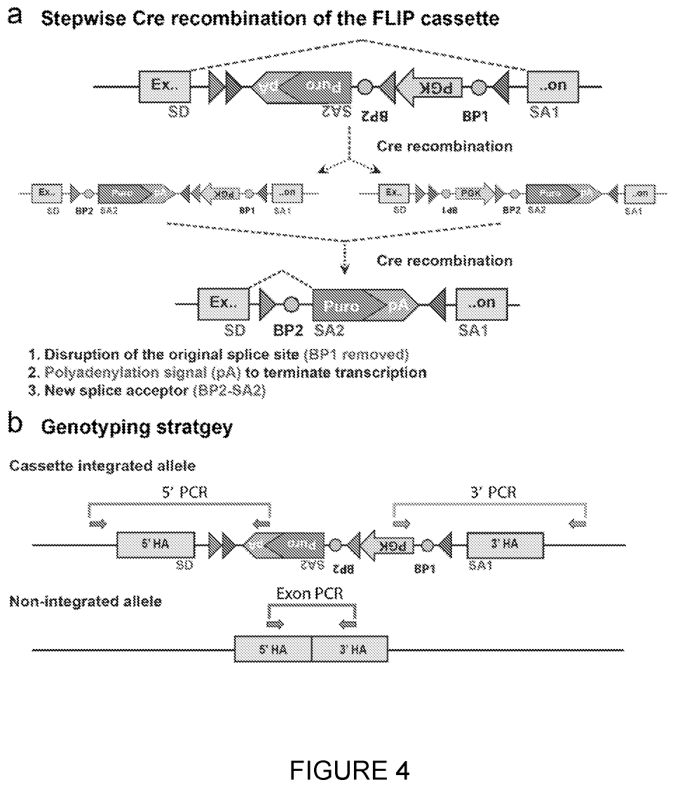

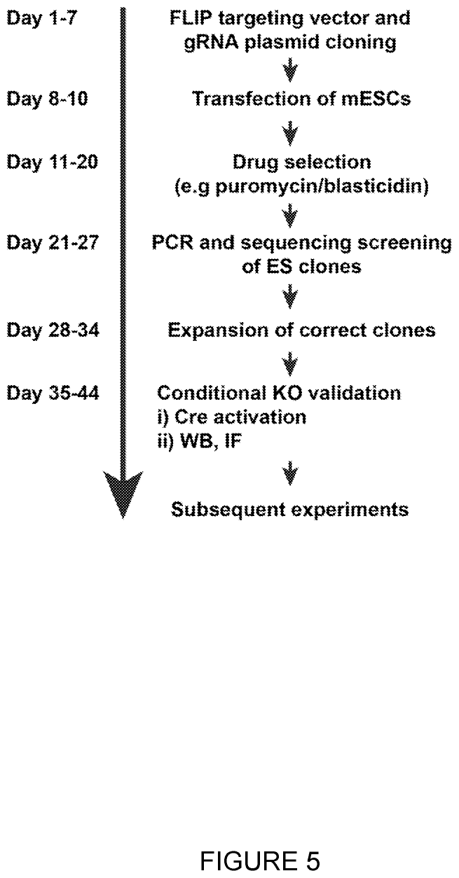

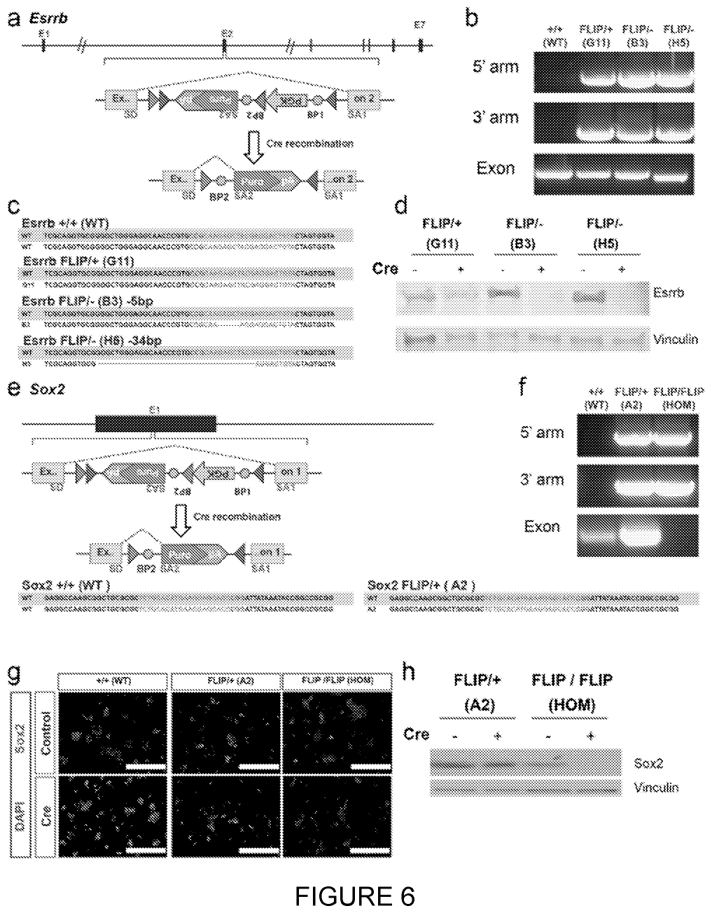

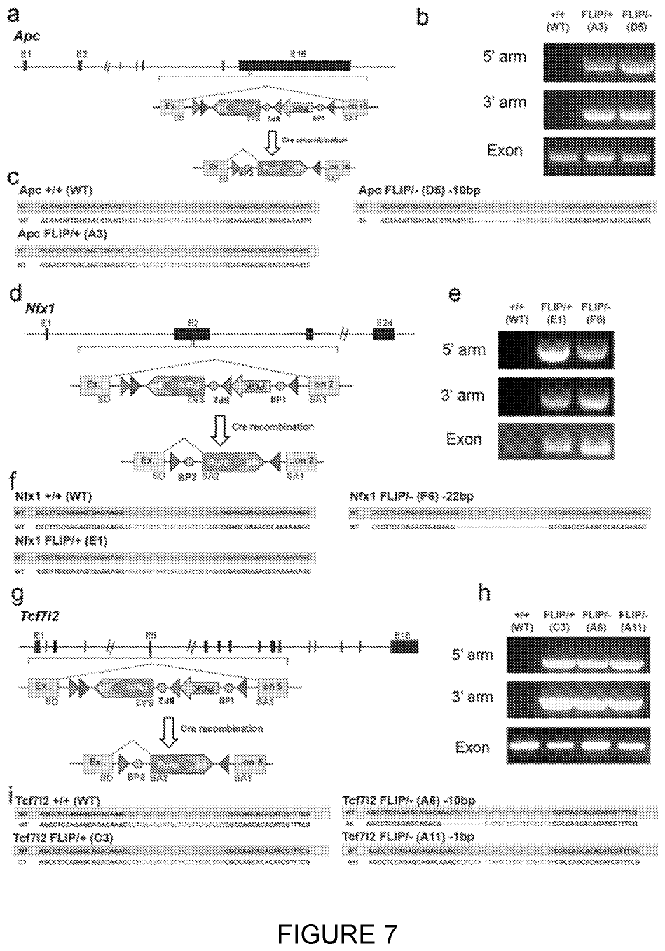

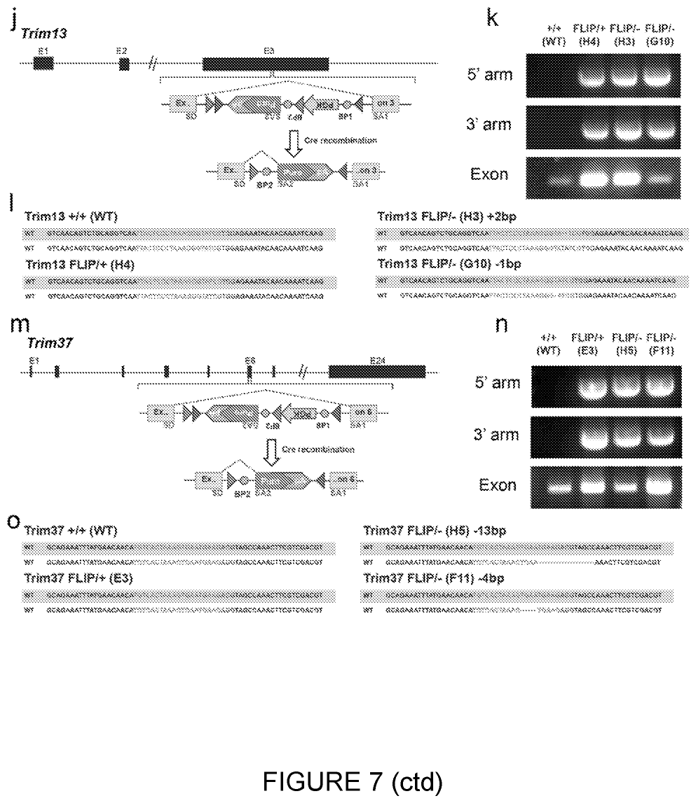

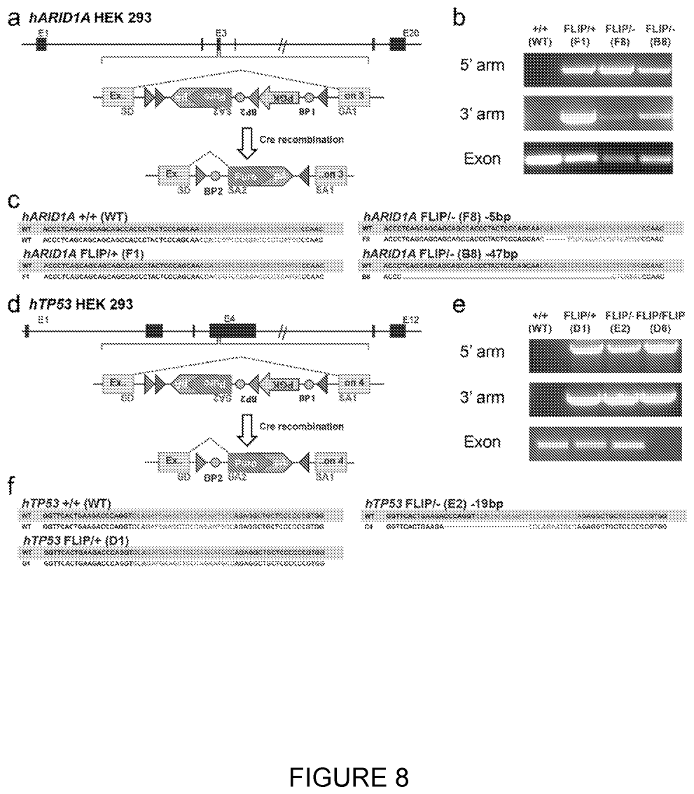

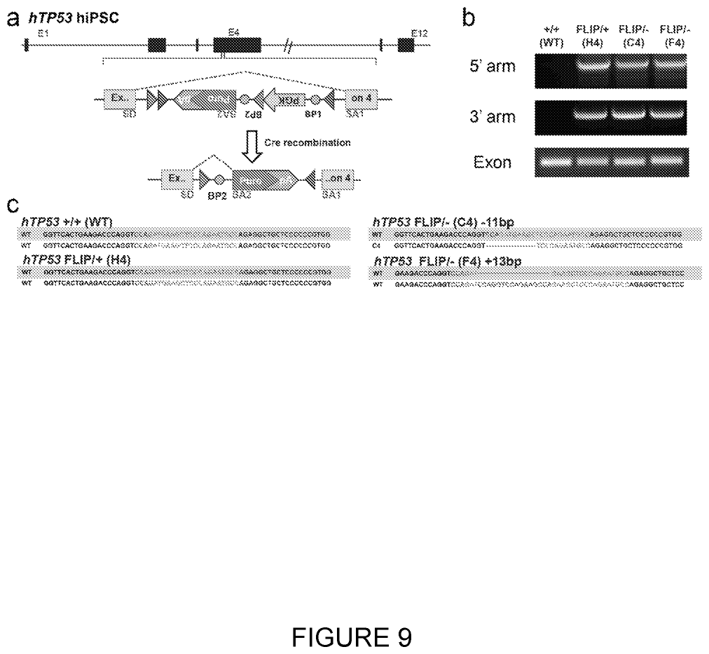

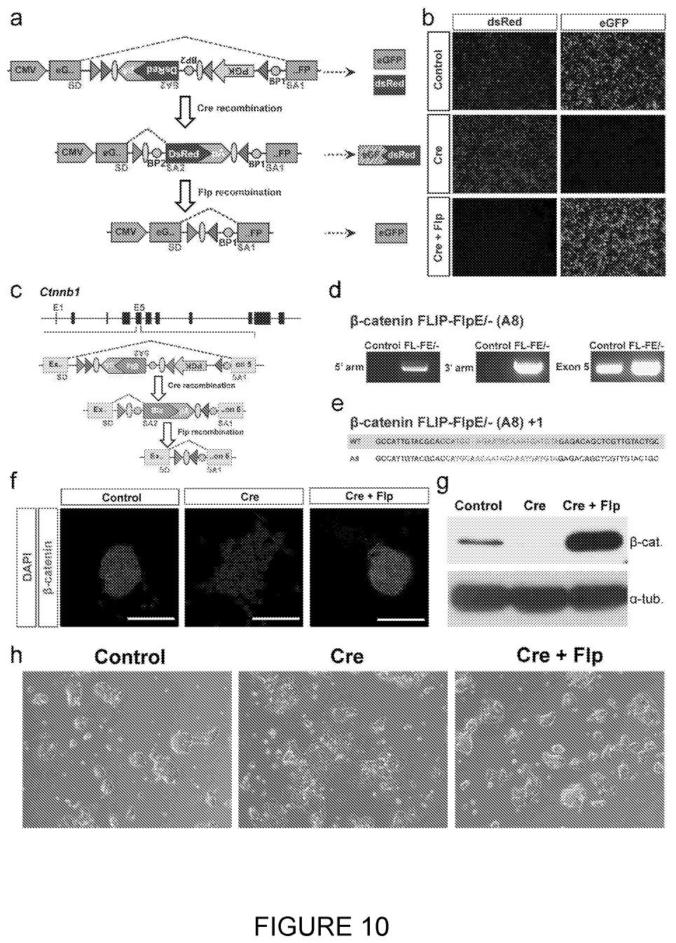

From 64 clones, 14 clones (21.9%) were correctly targeted, among which 4 clones carried a frame-shift mutation in the second allele (FIG. 3). The recovery of β-catenin compound mutant clones (FLIP targeted/NHEJ frameshift; FLIP/−) with wild type morphology strongly suggests that the insertion of the FLIP cassette does not disrupt the function of β-catenin in the non-mutagenic orientation. Upon expression of Cre recombinase in FLIP/−clones, we observed a loss of β-catenin expression in cells (FIG. 2d, e). Moreover, compared to control (FLIP/+) cells treated with Cre recombinase, the FLIP/−cells became scattered and lost their dome-like morphology (FIG. 2f).

To test if the CRISPR-FLIP technology is widely applicable, we additionally targeted Apc, Esrrb, Nfx1, Sox2, Tcf7/2, Trim13, and Trim37 in mESCs; TP53 and ARID1A in human HEK293 cells; and TP53 in human induced pluripotent stem cells (FIG. 6-9). The FLIP intron targeting efficiency ranged from 19.8% to 40.6% in mESCs (FIG. 3). For all genes, FLIP/−clones were obtained (FIG. 3, 6-9). The conditional inactivation of gene expression was confirmed by Western blot and immunofluorescence for Esrrb and Sox2 (FIG. 6). The inventors conclude that the FLIP conditional strategy is efficient and can be applied widely for conditional loss-of-function studies in various mammalian cells that are amenable to Cas9-assisted gene targeting.

The strategy presented herein requires the presence of a CRISPR site overlapping or nearby the insertion site of the FLIP cassette, imposing constraints on the exons than can be targeted. To maximize the potential for a null mutation, the target exon must be common to all transcripts and lie within the first 50% of the protein-coding sequence. Additionally, based on the minimum size of mammalian exons (50 bp) (Dominski, Z. & Kole, R. (1991) Mol. Cell. Biol. 11, 6075-83), we set the size of the split exons to be at least 60 bp. Finally, for optimal splicing, we chose insertion points that match the consensus sequence for mammalian splice junctions (minimally MAGR (^{A}/_{C}AG/Pu)) (Stephens, R. M. & Schneider, T. D. (1992) J. Mol. Biol. 228(4), 1124-36). Using this set of rules, we used bioinformatics to estimate the number of suitable FLIP insertion sites in the protein-coding genes in the mouse and human genomes. Our bioinformatics analysis revealed 1,171,712 FLIP insertion sites and corresponding gRNA binding sites covering 16,460 genes in the mouse genome and 1,171,787 FLIP insertion sites and corresponding gRNA binding sties covering 15,177 genes in the human genome.

Here the inventors present the FLIP technology, a method that allows one-step generation of bi-allelic conditional gene modifications using only a single gRNA, Cas9 and a simple donor vector. Compared to the conventional strategies for the generation of conditional alleles, the FLIP cassette, when combined with the CRISPR/Cas9, enables highly efficient bi-allelic conditional gene modification in a single round of gene targeting without the need to remove the drug selection cassette. The FLIP targeting vectors only require short homologous arms (less than 1 kb) which makes the assembly of targeting vectors easy and scalable. The FLIP cassette is invariable and can be generically applied to any gene, including non-coding RNA genes.

### EXAMPLE 3

### Reversible Conditional Modification

The inventors further modified the FLIP intronic cassette to generate a reversible conditional allele. The region containing the cryptic splice acceptor and pA is flanked by two FRT sites (FIG. 10a, FLIP-FIp Excision (FLIP-FIpE)). When inserted into eGFP, the intronic FLIP-FIpE cassette permits the expression of eGFP. Upon Cre recombination the FLIP-FIpE cassette turns into the mutagenic orientation, like the FLIP cassette, which blocks the eGFP expression. Next, the added FRT sites enables the mutagenic FLIP-FIpE cassette to be excised by FIp recombinase, thus allowing the revival of eGFP expression (FIG. 10a). The FLIP-FIpE cassette was inserted in the 5^{th} exon of the mouse β-catenin allele. The Ctnnb1^{FLIP-FIpE/-} (FLIP-FIpE targeted/NHEJ frameshift; FLIP-FIpE/−) mutant clones went through the series of recombination, first by Cre and then FIp. At each step, the mutant showed wildtype, mutant (after Cre), and again wildtype (after Cre and FIp) morphology, respectively (FIG. 10b). Accordingly, a loss and a gain of β-catenin expression in cells was observed (FIG. 10c,d), indicating that the FLIP intronic cassette can also be used for ‘switchable’ gene expression with a simple modification.

1. A method for bi-allelic conditional modification of a target gene, comprising:
providing a nucleic acid construct that is an artificial intron comprising
(a) an expression cassette in antisense orientation relative to the target gene;
(b) one or more pairs of recombinase sites, wherein at least one pair flanks the expression cassette; and
(c) one or more components that inactivate the target gene, and exposing a target gene to the nucleic acid construct in the presence of a recombinase, whereby the expression cassette inverts and the target gene is inactivated.

2. The method of claim 1, wherein the bi-allelic conditional modification of a target gene is reversible.

3. A nucleic acid construct which is an artificial intron comprising a splice donor at one end, a first branch point and a first splice acceptor at the other end, the construct comprising:
(a) an expression cassette positioned between the splice donor and first branch point, said expression cassette comprising a promoter, an open reading frame and a 3′ untranslated region, each of which is in antisense orientation relative to the first splice donor, branch point and splice acceptor;
(b) a first pair of recombinase sites, the first of which is positioned between the splice donor and the 3′ untranslated region of the expression cassette and the second of which is positioned between the first splice acceptor and the first branch point;
(c) a second branch point and second splice acceptor, each of which is positioned between the promoter and open reading frame of the expression cassette and is in antisense orientation relative to the first splice donor, branch point and splice acceptor; and
(d) a second pair of recombinase sites which flank the open reading frame, 3′ untranslated region, second splice acceptor and second branch point,
wherein following exposure to a recombinase, the orientation of said first pair of recombinase sites causes inversion of said expression cassette and results in one recombinase site from the first pair of recombinase sites and one recombinase site from the second pair of recombinase sites being orientated to cause excision of the promoter and first branch point.

4. The nucleic acid construct of claim 3, wherein the open reading frame encodes one or more selectable markers.

5. The nucleic acid construct of claim 4, wherein the open reading frame comprises a drug resistance gene.

6. The nucleic acid construct claim 3, wherein said nucleic acid construct additionally comprises:
(e) a third pair of recombinase sites, wherein said third pair of recombinase sites are distinct from the first and second pair of recombinase sites and flank the open reading frame and 3′ untranslated region of the expression cassette, second splice acceptor and second branch point,
wherein following exposure to a recombinase, the orientation of said third pair of recombinase sites causes excision of the open reading frame and 3′ untranslated region of the expression cassette, second splice acceptor and second branch point.

7. The nucleic acid construct of claim 6, wherein the third pair of recombinase sites comprise FRT sites.

8. The nucleic acid construct claim 3, wherein the 3′ untranslated region comprises a transcriptional termination signal, such as a polyadenylation signal.

9. The nucleic acid construct of claim 3, wherein following exposure to a recombinase said second branch point and second splicing acceptor are orientated to cause productive splicing with the first splice donor.

10. The nucleic acid construct of claim 3, which is downstream of a promoter and/or within a reporter gene.

11. A method for conditional gene modification, comprising: providing the nucleic acid construct of claim 3.

12. A method for reversible conditional gene modification, comprising: providing the nucleic acid construct of claim 6.

13. The method of claim 12, wherein said conditional gene modification is bi-allelic.

14. A method of conditional gene modification, comprising:
(a) co-transfection of a double-strand break-inducing agent, a gene targeting agent and the nucleic acid construct as defined in claim 3 into a cell;
(b) selection of a cell wherein at least one allele comprises the nucleic acid construct; and
(c) exposing the cell as defined in step (b) to a recombinase specific for the first and/or second pair of recombinase sites.

15. The method for reversible gene modification, comprising the method of claim 14, further comprising:
(d) exposing the cell to a further recombinase specific for the third pair of recombinase sites.

16. The method of claim 14, wherein the selection as defined in step (b) is of a cell wherein the first allele comprises the nucleic acid construct and the second allele comprises a gene-inactivating mutation and/or the nucleic acid construct.

17. The method of claim 14 , wherein said gene targeting agent is gRNA.

18. The method of claim 14, wherein said double-strand break inducing agent is selected from TALENs, zinc finger nucleases and Cas9.

19. The method of claim 18, wherein said double-strand break inducing agent is Cas9.

20. The method of claim 14, wherein said selection comprises use of the expression cassette and/or polymerase chain reaction and/or sequencing.

21. The method of claim 14, wherein the gene inactivating mutation is an indel-mediated frameshift or truncation mutation.

22. The method of claim 21, wherein the indel is a product of non-homologous end joining.


SEQUENCE LISTING (17 sequences)

SEQ ID NO: 1 - DNA, 14 bp, Artificial
  Splice acceptor
tttccctcccttag

SEQ ID NO: 2 - DNA, 505 bp, Artificial
  Promoter
cccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgac

SEQ ID NO: 3 - DNA, 678 bp, Artificial
  Open reading frame
atggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtga

SEQ ID NO: 4 - DNA, 600 bp, Artificial
  Open reading frame
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 5 - DNA, 354 bp, Artificial
  Transcriptional termination signal
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcacttgttaattgcagcttataatggttacaaataaagcaatagcatcacaaatttcacaaataaagcatttttttcactgcattctagttgtggtttgtccaaactcatcaatgtatcttatcatgtct

SEQ ID NO: 6 - DNA, 1931 bp, Artificial
  Nucleic acid construct
gtaagtatcaaagatctggcgcgccataacttcgtatagtacacattatacgaagttatggctaagctttgtgccatgccattgatgctagcacagcctgaatggataacttcgtataggataccttatacgaagttatgtcgacagacatgataagatacattgatgagtttggacaaaccacaactagaatgcagtgaaaaaaatgctttatttgtgaaatttgtgatgctattgctttatttgtaaccattataagctgcaattaacaagtgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacatcaggaattcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtacccacctgagggagggaaaatagaccaataggcagagagagtcagtgcctatcagaaacccaagagtcttctctgtctccacgtgcccagtttctattggtctccttaaacctgtcttgtaacctctagataacttcgtataatgtgtactatacgaagttatcgatggctgtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccgggtgaggcggccgcggttacaagacaggtttaaggagaccaatagaaactgggcatgtggagacagagaagactcttgggtttctgataggcactgacataacttcgtataaggtatcctatacgaagttattttccctcccttag

SEQ ID NO: 7 - DNA, 2008 bp, Artificial
  Nucleic acid construct
gtaagtatcaaagatctggcgcgccataacttcgtatagtacacattatacgaagttatggctaagctttgtgccatgccattgatgctagcacagcctgaatggataacttcgtataggataccttatacgaagttatgtcgacagacatgataagatacattgatgagtttggacaaaccacaactagaatgcagtgaaaaaaatgctttatttgtgaaatttgtgatgctattgctttatttgtaaccattataagctgcaattaacaagtgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacatcaggaattcactggaacaggtggtggcgggcctcggcgcgctcgtactgctccaccacggtgtagtcctcgttgtgggaggtgatgtccagcttggagtccacgtagtagtagccgggcagcttcacgggcttcttggccatgtagattgacttgaactccaccaggtagtggccgccgcccttcagcttcagcgccttgtggatctcgcccttcagcacgccgtcgcgggggtacaggcgctcggtggagggctcccagcccagagtcttcttctgcattacggggccgtcggaggggaagttcacgccgatgaacttcacgtggtagatgaaggtgccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcacctgcagcttggcggtctgggtgccctcgtagggcttgccctcgccctcgccctcgatctcgaactcgtggccgttcacggagccctccatgtgcaccttgaagcgcatgaagggcttgatgacgttctcagtgctatccatggtacccacctgagggagggaaaatagaccaataggcagagagagtcagtgcctatcagaaacccaagagtcttctctgtctccacgtgcccagtttctattggtctccttaaacctgtcttgtaacctctagataacttcgtataatgtgtactatacgaagttatcgatggctgtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccgggtgaggcggccgcggttacaagacaggtttaaggagaccaatagaaactgggcatgtggagacagagaagactcttgggtttctgatagcactgacataacttcgtataaggtatcctatacgaagttattttccctcccttag

SEQ ID NO: 8 - DNA, 5163 bp, Artificial
  Nucleic acid construct in vector
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgtgtgaagagcgcgatcgcgtttaaacgctcttcagtaagtatcaaagatctggcgcgccataacttcgtatagtacacattatacgaagttatggctaagctttgtgccatgccattgatgctagcacagcctgaatggataacttcgtataggataccttatacgaagttatgtcgacagacatgataagatacattgatgagtttggacaaaccacaactagaatgcagtgaaaaaaatgctttatttgtgaaatttgtgatgctattgctttatttgtaaccattataagctgcaattaacaagtgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacatcaggaattcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtacccacctgagggagggaaaatagaccaataggcagagagagtcagtgcctatcagaaacccaagagtcttctctgtctccacgtgcccagtttctattggtctccttaaacctgtcttgtaacctctagataacttcgtataatgtgtactatacgaagttatcgatggctgtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccgggtgaggcggccgcggttacaagacaggtttaaggagaccaatagaaactgggcatgtggagacagagaagactcttgggtttctgataggcactgacataacttcgtataaggtatcctatacgaagttattttccctcccttagtgaagagcgtttaaacgcgatcgcgctcttcataagacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 9 - DNA, 7898 bp, Artificial
  Nucleic acid construct in vector
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggtaagtatcaaagatctggcgcgccataacttcgtatagtacacattatacgaagttatggctaagctttgtgccatgccattgatgctagcacagcctgaatggataacttcgtataggataccttatacgaagttatgtcgacagacatgataagatacattgatgagtttggacaaaccacaactagaatgcagtgaaaaaaatgctttatttgtgaaatttgtgatgctattgctttatttgtaaccattataagctgcaattaacaagtgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacatcaggaattcactggaacaggtggtggcgggcctcggcgcgctcgtactgctccaccacggtgtagtcctcgttgtgggaggtgatgtccagcttggagtccacgtagtagtagccgggcagcttcacgggcttcttggccatgtagattgacttgaactccaccaggtagtggccgccgcccttcagcttcagcgccttgtggatctcgcccttcagcacgccgtcgcgggggtacaggcgctcggtggagggctcccagcccagagtcttcttctgcattacggggccgtcggaggggaagttcacgccgatgaacttcacgtggtagatgaaggtgccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcacctgcagcttggcggtctgggtgccctcgtagggcttgccctcgccctcgccctcgatctcgaactcgtggccgttcacggagccctccatgtgcaccttgaagcgcatgaagggcttgatgacgttctcagtgctatccatggtacccacctgagggagggaaaatagaccaataggcagagagagtcagtgcctatcagaaacccaagagtcttctctgtctccacgtgcccagtttctattggtctccttaaacctgtcttgtaacctctagataacttcgtataatgtgtactatacgaagttatcgatggctgtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccgggtgaggcggccgcggttacaagacaggtttaaggagaccaatagaaactgggcatgtggagacagagaagactcttgggtttctgatagcactgacataacttcgtataaggtatcctatacgaagttattttccctcccttaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagactacaaggacgacgatgacaaggctggagcagactacaaggacgacgatgacaagctcgatggaggatacccctacgacgtgcccgactacgccgctggagcatacccctacgacgtgcccgactacgcctgatcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 34 bp, Artificial
  Recombinase site
ataacttcgtatagtacacattatacgaagttat

SEQ ID NO: 11 - DNA, 34 bp, Artificial
  Recombinase site
ataacttcgtataggataccttatacgaagttat

SEQ ID NO: 12 - DNA, 64 bp, Artificial
  Flip_UniL primer
gtgtgaagagcgcgatcgcgtttaaacgctcttcagtaagtatcaaagatctggcgcgccataa

SEQ ID NO: 13 - DNA, 93 bp, Artificial
  Flip_UniR primer
ttatgaagagcgcgatcgcgtttaaacgctcttcactaagggagggaaaataacttcgtataggataccttatacgaagttatgtcagtgcct

SEQ ID NO: 14 - DNA, 39 bp, Artificial
  Puro-L-Acc651 primer
ccctcaggtgggtaccatgaccgagtacaagcccacggt

SEQ ID NO: 15 - DNA, 31 bp, Artificial
  Puro-R-EcoRI primer
ggcacatcaggaattcaggcaccgggcttgc

SEQ ID NO: 16 - DNA, 41 bp, Artificial
  Blast-L-Acc651 primer
ccctcaggtgggtaccatggccaagcctttgtctcaagaag

SEQ ID NO: 17 - DNA, 40 bp, Artificial
  Blast-R-EcoRI primer
ggcacatcaggaattcagccctcccacacataaccagagg